(19) United States
(12) Patent Application Publication
(10) Pub. No.: US 2010/0042122 A1
(43) Pub. Date: Feb. 18, 2010

(54) EAR CURETTE WITH TRIANGULAR HANDLE

(76) Inventor: John K. Shaw, JR., Oxford, CT (US)

Correspondence Address: HOLLAND & BONZAGNI, P.C., 171 DWIGHT ROAD, SUITE 302, LONGMEADOW, MA, 01106-1700, US

(21) Appl. No.: 12/539,094

(22) Filed: Aug. 11, 2009

Related U.S. Application Data
(60) Provisional application No. 61/088,171, filed on Aug. 12, 2008.

(51) Int. Cl. A61F 11/00

(52) U.S. Cl. 606/162

(57) ABSTRACT

Applicant has disclosed an improved ear curette for use by medical practitioners, and other healthcare professionals, to remove cerumen (a.k.a. earwax). In the preferred embodiments, the curette comprises: an elongated “no-roll” handle which is substantially triangular in cross-section, with three round “edges,” along substantially the entire length of the handle; and two differently sized collection ends, either paddles or loops, integral with and at opposite ends of the handle. Practitioners can grip the “triangular” handle much like a pencil, between their thumbs and forefingers. This gives practitioners precise control and feel during collection procedures to remove carefully earwax build-up or blockage from patients' ear canals.

### RELATED APPLICATION

This application claims priority from U.S. Provisional Patent Application Ser. No. 61/088,171, filed Aug. 12, 2008. Applicant incorporates that provisional application herein by reference.

### FIELD OF INVENTION

This invention relates generally to ear curettes. More specifically, it relates to ear curettes used by medical practitioners and other healthcare professionals to remove build-up or blockage in a patient's ear canal.

### BACKGROUND OF INVENTION

Cerumen is the yellow wax-like secretion from the glands of the external ear. It is also called earwax.

Cerumen impaction has the highest diagnosis in older adults, children, and patients with mental retardation. Cerumen removal is the most common ear, nose, and throat (“ENT”) procedure performed in primary care.

Medical practitioners, and other healthcare professionals, typically use ear curettes to remove the cerumen build-up or blockage in a patient's ear canal. Due to the potential for injury, it is important for a curette to be precisely handled during such a collection procedure, and with delicate control.

Many different types of ear curettes have been patented, such as: U.S. D275,127 to Edwards; U.S. D318,117 to Michelson; U.S. D423,669 to Huttner; U.S. D428,489 to Huttner; U.S. D445,503 to Huttner; U.S. Pat. No. 1,737,106 to Campbell et al.; U.S. Pat. No. 4,044,770 to Ocel et al.; U.S. Pat. No. 5,209,767 to Krug et al.; U.S. Pat. No. 5,586,989 to Bray, Jr.; and U.S. Pat. No. 7,074,230 to Olson.

Accordingly, it is a principal object of the present invention to provide an ear curette with an improved “no-roll” handle for better control of the curette by medical practitioners and other healthcare professionals.

It is a more specific object to provide an ear curette with a handle which is triangular in cross-section, wherein the handle allows for quick orientation and control by a practitioner.

It is a more specific object to provide a disposable ear curette, commensurate with the above-listed objects, which is inexpensive to manufacture, yet safe and durable to use.

### SUMMARY OF INVENTION

Applicant has disclosed an improved disposable ear curette having a substantially triangularly-shaped “no-roll” handle in cross-section, preferably with beveled edges. Its triangular shape allows for the quick and accurate orientation of the collection end(s) for optimal control, reach and collection by a medical practitioner. Applicant's preferred embodiments have two differently sized collection ends. The collection ends preferably are either paddles or loops.

Now health care professionals can utilize a single device for complete removal of impacted or occluded cerumen from the ear canal. The different size tips on one single device allows for the larger tip to be used to remove the substantial impaction areas and the smaller device to clean the remaining cerumen particles that are particularly hard to retrieve from the ear canal.

### BRIEF DESCRIPTION OF DRAWINGS

The above and other objects will become more readily apparent when the following description is read in conjunction with the accompanying drawings, in which:

FIG. 1 is a perspective view of a preferred embodiment of Applicant's “Ear Curette with Triangular Handle”;

FIG. 2 is a perspective view of a paddle collection end of the FIG. 1 curette;

FIG. 3 is a perspective view of another paddle collection end of the FIG. 1 curette;

FIG. 4 is a side plan view of the FIG. 1 curette;

FIG. 5 is a cross-sectional view taken along sight line 5-5 of FIG. 4;

FIG. 6 is a perspective view of an alternate embodiment of Applicant's “Ear Curette with Triangular Handle”;

FIG. 7 is a perspective view of a loop collection end of the FIG. 6 curette;

FIG. 8 is a perspective view of another loop collection end of the FIG. 6 curette;

FIG. 9 is a side plan view of the FIG. 6 curette; and

FIG. 10 is a cross-sectional view taken along sight line 10-10 of FIG. 9.

### DETAILED DESCRIPTION OF PREFERRED EMBODIMENTS

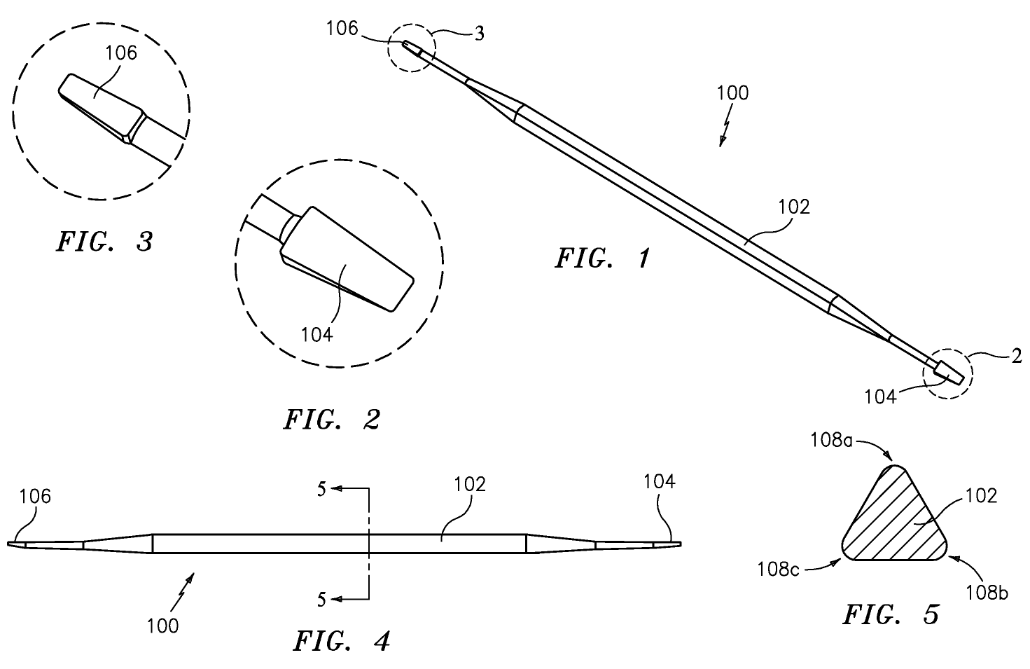

Referring to the drawings, Applicant has disclosed an improved ear curette for use by medical practitioners and other healthcare professionals for removal of cerumen buildup or blockage. In a preferred embodiment (see FIGS. 1-5), a curette 100, preferably made of plastic, comprises: an elongated handle 102 which is substantially triangular in cross-section (see FIGS. 1, 4, 5) along substantially its entire length; and two collection, ends 104, 106 at opposite ends of the handle. The depicted collection ends 104, 106 are paddles or scoops, with collection end 104 being larger than collection end 106. See FIGS. 2, 3.

Applicant's invention lies primarily in the shape of handle 102 rather than the shapes of collection ends 104, 106. The handle's triangular cross-sectional shape permits a medical practitioner to quickly and accurately orient the curette's collection ends for optimal control, reach and collection.

Handle 102 preferably has round longitudinal “edges” at 108a, 108b, 108c for smooth handling. It could be manufactured with straight edges, if desired, but that might be more difficult to manipulate.

A medical practitioner will grip the curette 100 much like a pencil and because of the triangular design will be able to orient the curette 100 between the practitioner's thumb and forefinger giving precise control and feel for the collection procedure.

The practitioner will use the larger collection end 104 for the initial removal of build-up or blockage, then will flip the device 180 degrees to use the smaller collection end 106 to complete the procedure and remove the remaining build-up or blockage. Hence, the collection ends 104, 106 can thought of singularly, and in combination, as removal means for removing impacted cerumen from a patient (not shown).

The triangularly-shaped handle 102 (see FIG. 5) will also prevent the device from moving or rolling away such as when it is on a tray or examination table. Hence, it is a “no-roll” handle.

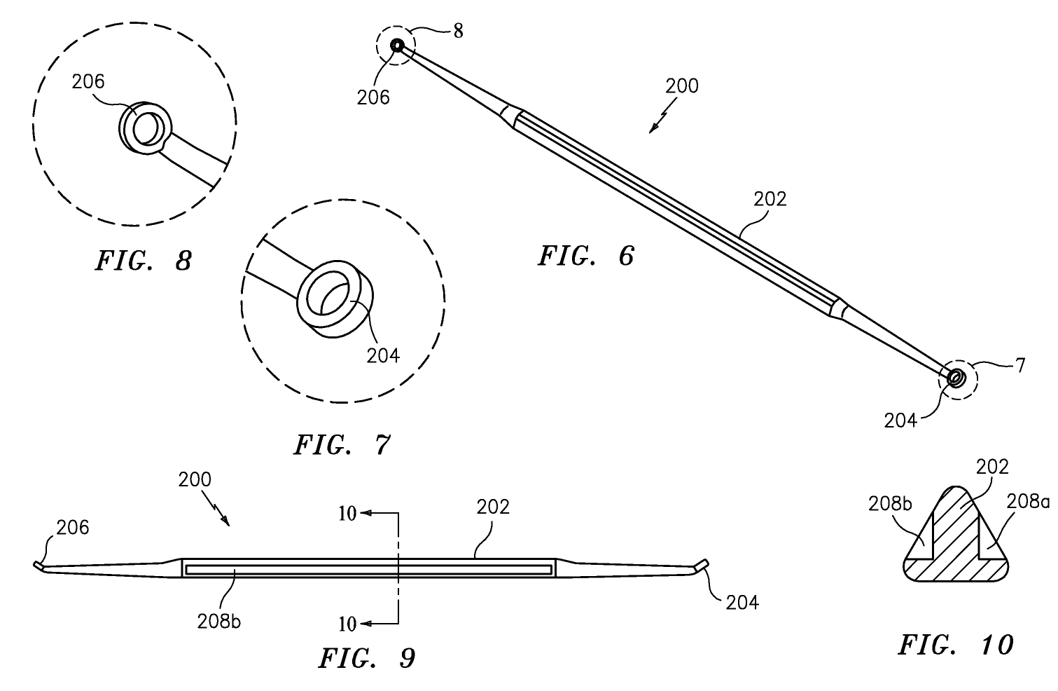

FIGS. 6-10 depict another preferred embodiment 200. It includes an elongated handle 202 with rings or loops, instead of paddles, as the collection ends 204, 206. Loops are commonly referred to as the “buck” style of curettes. Collection end 204 is larger than collection end 206.

Handle 202 contains elongated side notches 208a, 208b. The notches 208a, 208b serve a dual purpose: the main purpose is to control sink in the injection molding process and the other is to provide an enhanced grip.

Applicant's two preferred curette embodiments 100, 200, with differently sized ends, allow one curette to clean all areas of the ear cavity so that a practitioner can keep fewer varieties of devices on hand.

Other collection ends, besides those depicted, could be manufactured in a wide variety and combination, while maintaining the triangular-shaped handle.

Since the preferred curettes 100, 200 are made of extruded plastic, they are designed to be inexpensive and disposable. That minimizes the need to sanitize stainless steel curettes.

It should be understood by those skilled in the art that obvious structural modifications can be made without departing from the spirit or scope of the invention. For example: the curette 100 is preferably made of injection-molded plastic, but it could be manufactured from other materials using different processes, and still be easily disposable—one possible example is recyclable plastic or compressed paper; the curette could be made with only one collection end; and the device could be manufactured with Antimicrobial properties to lessen the risk of infection.

We claim:

1. An ear curette comprising:
a. an elongated handle which is at least substantially triangular in cross-section along substantially the entire length of the handle; and
b. two collection ends of the ear curette which are located at opposite ends of the handle.

2. The ear curette of claim 1 wherein the handle has three round edges along substantially the entire length of the handle.

3. The ear curette of claim 2 wherein the handle contains side finger notches.

4. The ear curette of claim 2 wherein the collection ends are differently sized.

5. The ear curette of claim 4 wherein the collection ends are paddles.

6. The ear curette of claim 5 wherein the collection ends are loops.

7. An ear curette comprising:
a. an elongated handle which is at least substantially triangular in cross-section along substantially the entire length of the handle; and
b. removal means for removing impacted cerumen, wherein the removal means comprises at least one collection end of the curette integrally attached to the handle.

8. The ear curette of claim 7 wherein the handle has three round edges along substantially the entire length of the handle.

9. The ear curette of claim 7 wherein the handle contains side finger notches.

10. The ear curette of claim 9 wherein the at least one collection end comprises two collection ends which are located at opposite ends of the handle.

11. The ear curette of claim 10 wherein the two collection ends are differently sized.

12. The ear curette of claim 11 wherein the two collection ends are paddles.

13. The ear curette of claim 11 wherein the two collection ends are loops.
